(19) United States
(12) Patent Application Publication
(10) Pub. No.: US 2002/0045171 A1
(43) Pub. Date: Apr. 18, 2002

(54) Method of profiling genes as risk factors for attention deficit hyperactivity disorder

(76) Inventor: David E. Comings, Duarte, CA (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/825,922

(22) Filed: Apr. 5, 2001

Related U.S. Application Data
(60) Provisional application No. 60/195,312, filed on Apr. 10, 2000.

(51) Int. Cl. C12Q 1/68

(52) U.S. Cl. 435/6

(57) ABSTRACT

The present invention relates to methods of profiling candidate genes as risk factors for attention deficit hyperactivity disorder, oppositional defiant disorder and conduct disorder. In one embodiment, the invention relates to a method of determining a genetic predisposition of a subject to ADHD, comprising detecting at least one allele from the group comprising the TPH, PNMT, ADO42A, NOS3, and NAT1 genes. By focusing on the additive effect of multiple genes and on the cumulative effect of functionally related groups of genes, a powerful approach is provided for the dissection of the genetic basis of ADHD, ODD and CD.

### CROSS REFERENCE TO RELATED APPLICATIONS

This application is related to provisional application Serial No. 60/195,312, filed Apr. 10 , 2000, which is incorporated herein by reference.

### BACKGROUND OF THE INVENTION

Attention deficit hyperactivity disorder is the most common behavioral disorder affecting children. Recent twin studies have indicated that 75 to 90 percent of the variance of ADHD is attributable to additive genetic factors (Stevenson 1992; Stevenson 1993; Sherman et al. 1997a; Sherman et al. 1997b, Gillis et al. 1992; and Hudziak et al. 1997). The publications and other materials used herein to illuminate the background of the invention or provide additional details respecting its practice, are incorporated by reference, and for convenience are respectively grouped in the appended Lists of References. Some of the specific genes involved are just beginning to be identified. They include the dopamine D₂ receptor (DRD2) (Comings et al. 1991; 1996a), dopamine D₄ receptor (DRD4) (Lahoste et al. 1996; Swanson et al. 1998; Rowe et al. 1998; Faraone et al. 1998), dopamine D₅ receptor (DRD5) (Daly et al. 1999), dopamine transporter (DAT1) (Cook et al. 1995; Comings et al. 1996a; Gill et al. 1997; Waldman et al. 1996; Daly et al. 1999), dopamine β-hydroxylase (Comings et al. 1996a; Daly et al. 1999), adrenergic α2A (Comings et al. 1999a), adrenergic α2C (Comings et al. 1999b), and monoamine oxidase A (Comings et al. 1999a). Some studies for these genes have been negative (Lau et al. 1997).

Most complex behavioral disorders are inherited as polygenic traits interacting with the environment. A major characteristic of polygenic inheritance is that multiple genes are involved with each gene contributing a small effect. The effects can be additive, heterotic (over-dominant) or epistatic. While numerous examples of epistasis have been reported, the additive effects and heterotic effects are considered to be predominant (Falconer 1981; Loehlin 1992; Lynch 1998). A phenotypic effect is obtained when an individual inherits enough of these genes to exceed a threshold that puts them at increased risk of expressing a given trait (Falconer 1965). The major implications of such a model are that studies that examine one gene at a time provide an incomplete picture, and tend to produce a series of both successful and unsuccessful attempts at replication. The present study is based on the assumption that the most effective way to identify the individual genes and groups of genes for disorders that are due to the additive effect of multiple genes is to study the additive effect of multiple candidate genes.

Genes of interest for ADHD, ODD and CD include the dopamine D₂ receptor gene (DRD2), the dopamine β-hydroxylase gene (DBH), the dopamine transporter gene (DAT 1) (Comings et al. 1 996a), adrenergic genes such as DBH, ADRA2A and ADRA2C (Comings et al. 1999a). Other genes of interest include genes for enzymes and receptors for other neurotransmitters (GABA, opioids, cannabinoids, adenosine, nitric oxide, acetyl choline), for hormones and neuropeptides, and for genes associated with immune functions (cd8, gamma interferon).

Thus, there is a continued need to develop new methods to examine the profiles of groups of genes as risk factors for phenotypic traits associated with complex nervous disorders such as ADHD, ODD and CD.

There is also a continued need to develop new methods to examine the correlation between individual genetic scores and any given phenotypic trait. Such approaches would allow the determination of the percent of the variance accounted for by any genes under examination and the significance of the correlation.

There is also a continued need to develop new methods to determine which groups of genes, for example the noradrenergic genes, play the greatest role in complex disorders such as ADHD,ODD and CD.

### SUMMARY OF THE INVENTION

The present invention is directed to methods of profiling genes as risk factors for attention deficit hyperactivity disorder, oppositional defiant disorder, and conduct disorder, and to the discovery that based on genetic profiling, the noradrenergic genes play the greatest role in ADHD.

In one aspect, the present invention is directed to a method of determining a genetic predisposition of a subject to ADHD, ODD and/or CD, comprising detecting at least one allele from the group comprising the DRD3, DRD4, HTR1DA, HTR6, TPH, ADRA2B, PNMT, GABBR1 , ADOA2A, GRIN2B, NOS3, MME, APN, NAT1, CRH, CCK, CYP, SBP, ESR, INS, OTR, CD8, INFG and PS1 genes.

In a second aspect, the present invention is directed to a method of examining the correlation between individual genetic scores and any given phenotypic trait, and determining the percent of the variance accounted for by any gene under examination as well as the significance of the correlation.

In a third aspect, the present invention is directed to a method for developing a polygenic assay that is diagnostic, comprising the steps of (a) identifying the trait that is to be studied; (b) creating a scale measuring the severity of the trait to be studied; (c) selecting at least one candidate gene that may contribute to said trait; (d) identifying at least one polymorphism associated with said candidate gene; (e) correlating allelic patterns of said polymorphism with said scale; (f) comparing the association of said allelic pattern to the correlation of said candidate gene to said trait; and (g) wherein the allelic patterns that are positively associated with said trait are added, to form a polygenic assay that is diagnostic.

In a fourth aspect of the invention, analysis of the profiles of forty-two genes (and polymorphisms thereof) identified herein is further provided for diagnosis of subjects with attention deficit hyperactivity disorder, oppositional defiant disorder, and conduct disorder. The diagnostic method comprises analyzing the DNA sequences of the genes under examination of an individual to be tested and comparing it with the DNA sequence of the native, non-variant genes. In a second embodiment, the genes of an individual to be tested are screened for polymorphisms associated with attention deficit hyperactivity disorder, oppositional defiant disorder, and conduct disorder. The ability to predict ADHD, ODD and CD will enable physicians to treat these disorders with appropriate medical therapies.

In a fifth aspect of the present invention, the genetic profiles of the suspect genes are used for drug screening and testing.

In a sixth aspect of the present invention, the present invention is directed a method of determining whether a given disorder is preferentially associated with the genes for hormones or neuropeptides.

### BRIEF DESCRIPTION OF THE FIGURES

FIGS. 1(a) and (b) describe the ANOVA of ADHD scores for the genotypes of forty genes.

FIG. 2 describes the final results for forty-two genes for the ADHD, ODD and CD traits.

FIG. 3 summarizes the results for individual genes that were included in the regression equation after all forty-two genes were entered as independent variables and the nonsignificant ones removed by backward elimination.

FIG. 4 summarizes the cumulative r² values for eight different groups of genes. The p values for the significant groups are shown.

### DETAILED DESCRIPTION OF THE INVENTION

The invention, in part, relates to a method of profiling genes as risk factors for attention deficit hyperactivity disorder (ADHD), oppositional defiant disorder (ODD) and conduct disorder (CD). An aspect of the invention utilizes a technique termed Multivariate Analysis of Associations to discover the relative role of forty-two different genes associated with ADHD, ODD and CD by multivariate regression analysis using a quantitative score for ADHD, ODD or CD as the dependent variables and backward elimination of the genes as independent variables. In another aspect of the invention, the correlation coefficient was determined between individual gene scores and phenotypic traits, and the scores of groups of genes were combined to create genetic profiles of multiple genes and associated risk assessments. See Comings et al. 2000a and Comings et al. 2000c, both of which are specifically incorporated herein by reference.

In order to study the concept polygenic inheritance above a certain threshold that increases the risk of an individual expressing a given trait, we examined the additive effect of forty-two genes on a range of phenotypic traits, including ADHD in Tourette syndrome probands and controls. The genes include the dopamine D₂ receptor gene (DRD2), the dopamine β-hydroxylase gene (DBH), the dopamine transporter gene (DAT 1), and three adrenergic genes, DBH, ADRA2A and ADRA2C. We also studied genes for enzymes and receptors for other neurotransmitters (GABA, opioids, cannabinoids, adenosine, nitric oxide, acetyl choline), for hormones and neuropeptides, and for genes associated with immune functions (cd8, gamma interferon). Because our results showed an association between the short alleles of the trinucleotide repeats of the androgen receptor gene with externalizing and aggressive behaviors (Comings et al. 1999b), we also tested the potential role of other hormone and neuropeptide genes, especially in CD.

Each gene was scored on the basis of whether the individual carried the genotype that was associated with a given phenotype (score =1), or was not associated with the phenotype (score=0). When the gene scores for each individual were added to form a composite polygenic (PG) score, that score could range from zero to three. The magnitude of quantitative traits for subjects carrying none, one, two, or all three of the genetic variants was examined by linear ANOVA. For the dopaminergic and adrenergic genes there was a significant increase in the magnitude of the traits in individuals carrying increasing numbers of the relevant gene variants. In a similar fashion, Pastinen et al. (1998) analyzed common variants of eight genes implicated previously as risk factors for coronary heart disease. They identified two independent genes that were additive in their effect such that the odds ratio was 4.5 (p=0.001) for individuals carrying 3 or 4 of the risk-increasing alleles compared to those carrying zero or one such allele. Noble et al (1998) reported an additive effect of the DRD2 and GABRB3 genes on alcoholism. When severe alcoholics were compared to controls the odds ratio was 8.0 for the DRD2 Taq I A1 allele, 4.2 for alleles of a repeat polymorphism of the GABRB3 gene, and 36.9 for those carrying the risk alleles of both genes.

As we began to elaborate on this concept by adding more genes, we found that a superior approach was to examine the correlation coefficient, r, between the individual gene scores and any phenotypic trait. This approach allowed the determination of r² or the percent of the variance accounted for by the genes being examined, F, and the p value or significance of the correlation. Since all analyses were reduced to r² with a constant range from 0 to 1, this approach allowed for the comparison of quantitative traits of varying magnitudes. The cumulative effect of multiple genes could be determined based on the magnitude of r².

Issues in studying multiple genes. Studies of the combined effect of multiple genes raise a number of issues that may not be encountered when genes are examined singly. These issues are the following: 1) scoring of the genes, 2) the relative versus the absolute effect of different individual genes and functional sets of genes, 3) the use of neutral polymorphisms, 4) the handling of X-linked genes, 5) the method of statistical analysis, and 6) analysis of comorbid disorders.

1. Scoring of the genes. In order to determine r² using linear regression analysis, it was necessary to score each gene according to the relative effect of the genotypes on the phenotype in question. To incorporate all the potential genotypes, based on the presence of two alleles (i.e. genotypes 11, 12, and 22), the range of the gene scoring was increased from 0 to 1 in the studies cited above, to 0 to 2. The score for the 11 genotype is on the left, the score for the 12 genotype is in the center, and the score for the 22 genotype is on the right. Thus, if the 11 genotype is associated with the lowest phenotype score, the 12 genotype with an intermediate score, and the 22 genotype with the highest genotype score, the three genotype scores would form a total gene score of 012 (see 2 allele codominant in Table 1).

TABLE 1
The 12 types of possible gene scores based on 0-2 range,
assuming two alleles or allele groups

| | 11 | 12 | 22 |
|---|---|---|---|
| Dominant | | | |
| 1 allele dominant | 2 | 2 | 0 |
| 2 allele dominant | 0 | 2 | 2 |
| Codominant | | | |
| 1 allele codominant | 2 | 1 | 0 |
| 2 allele codominant | 0 | 1 | 2 |
| Recessive | | | |
| 1 allele recessive | 2 | 0 | 0 |
| 2 allele recessive | 0 | 0 | 2 |
| Positive heterosis | | | |
| full positive heterosis | 0 | 2 | 0 |
| partial pos. hetero. 1 | 1 | 2 | 0 |
| partial pos. hetero. 2 | 0 | 2 | 1 |
| Negative heterosis | | | |
| full negative hetero. | 2 | 0 | 2 |
| partial neg. hetero. 1 | 2 | 0 | 1 |
| partial neg. hetero. 2 | 1 | 0 | 2 |

If a polymorphism had multiple alleles, they were placed into two allele groups and three genotype groups. We have previously presented the rationale for segregating microsatellite polymorphisms into larger (L) versus smaller (S) alleles in this fashion to form three genotype groups, SS, LS and LL (Comings 1998). Table 1 shows the twelve different types of possible gene scoring. The method of determining the scoring of the genes is a critical issue. In order to prevent problems with compounding chance effects, the most obvious method is for the scoring to be based on prior studies in the literature. For example, if several papers in the literature showed an association of the 11 and 12 genotypes of gene X with ADHD, the literature based gene score would be 220. However, there are a number of limitations of this approach: the results in the literature are often contradictory, the individuals studied may be of a different ethnic or racial background, the phenotypes studied may not be totally comparable, and most seriously, for many new genes there are no prior relevant studies. A second method that eliminates some of these problems is for the investigator to use one group of subjects to determine the method of coding the genes, and a second independent set of subjects to examine the combined effect of multiple genes. However, since both the initial and the re-test group may not have been evaluated by the same instrument, or since the number of subjects may be limited, this may not be a satisfactory solution.

2) The relative versus the absolute effect of different individual genes and functional sets of genes. The concern about the scoring of the genes is only an issue when the question is, “What is the absolute percent of the variance accounted for by a given gene or group of genes?” Since the method of scoring can have a major effect on the resultant r², scoring based on independent studies may be critical. However, a different question is, “What is the relative percent of the variance accounted for by a given gene, or a functionally related group of genes?” Here the effect of each gene is optimized if the method of scoring is based on assessment of the relative effect of the three genotypes by ANOVA, in the same set of subjects that are used for testing the composite effect of multiple genes. If each gene score is optimized in this fashion (optimized gene scoring), on average those genes that have the greatest physiologic effect will show the greatest relative impact on the phenotype compared to those genes with little or no effect. In other words, despite optimization of the gene scoring, and despite the impact of random results when the effect sizes are small, genes making little or no contribution to the genotype will tend to show a relatively smaller r² than those that contribute the most to the phenotype. Thus, when the issue is the examination of the relative effect of single genes or groups of genes, the sample used for gene scoring and the sample used for analyzing the combined effect of multiple genes, can be the same sample.

3. Neutral polymorphisms. A number of the polymorphisms used in this study were SNPs that did not involve functionally relevant portions of the respective genes. We have proposed that since polygenic disorders are common and due to the combined effect of multiple genes, the genetic variants involved are unique in that they must be very common and must have only a minor effect on gene function (Comings 1996; Comings 1998; Comings 1999c, Comings 1999d). We have also suggested that the varying lengths of the common microsatellite polymorphisms are responsible for much of this variation, through the formation of varying lengths of Z-DNA, which modulates gene function (Comings 1998). A corollary of this is that each gene may be associated with a wide range of hypo- and hyper-functional variants and that any common SNP, whether in a functional region of the gene or not, is likely to divide the gene into groups that vary in their phenotypic effect (Comings 1 999c).

4. Handling of X-linked genes. With experience, we found that unless the gender composition of the controls and subjects was virtually identical, the results for X-linked genes were likely to be inaccurate. Since this was not the case for this database, X-linked genes were excluded.

5) The method of statistical analysis. In the present invention we have used multivariate linear regression analysis with backward elimination to examine the composite effect of multiple genes. Three groups of genes were studied: six dopamine genes (DRD1, DRD2, DRD3, DRD4, DRD5, and DAT1), seven serotonin genes (HTT HTR1A, HTR1B, HTR1DA, HTR2A, TDO2 and TRH), and seven noradrenergic genes (DBH, ADRA2A, ADRAB, ADRA2C, PNMT, NET and COMT), other selected genes, and the total of all genes together. We term this a Multivariate Analysis of Associations (MAA) technique.

6) Analysis of comorbid disorders. A further advantage of this technique is that if the subjects are systematically evaluated at a range of phenotypes, especially those that are likely to be comorbid with the primary phenotype, the relative importance of individual genes or functional groups of genes can also be examined for those phenotypes. This allows testing of the hypothesis that comorbid conditions are present because they share some of the same genes (Comings 1996; Comings 1999d). We have examined CD and ODD, two conditions that are commonly comorbid with ADHD (Stewart et al. 1981; Faraone et al. 1991).

In summary, the comparison of the effect of multiple genes on a phenotypic variable taps into the major characteristics of polygenic inheritance—the additive, epistatic and heterotic effect of multiple genes. In addition it taps into the fact that while a number of different genes may be contributing to a given phenotype in the general population, in any given individual or group of individuals the effects of only a subset of those genes may be in evidence, i.e. genetic heterogeneity. The combination of small effect size and genetic heterogeneity often results in problems with replication when genes are studied singly. Examining and comparing the effect of multiple genes and functional groups of genes may compensate for the presence of different sets of genes in different individuals, with the same phenotype. Since the MAA technique can assess the effect of groups of genes, it may be more reproducible across different groups of subjects. To give a specific example, while variants of the DRD2 (Taq I A1 allele) (Comings et al. 1996), DRD4 (48 bp 7 repeat) (Lahoste et al. 1996; Swanson et al. 1998; Rowe et al. 1998; Faraone et al. 1998), DRD5 (Daly et al. 1999), and DAT1 (10 repeat allele) genes (Cook et al. 1995; Comings et al. 1996a; Gill et al. 1997; Waldman et al. 1996) have all been implicated in the etiology of ADHD, each may have a significant effect in one group of subjects but not in others. However, if the physiologic effect is similar for each gene (alteration in dopamine metabolism) the combined effect of all these genes might prove to more replicable across different groups of ADHD subjects, indicating it is a global dysregulation of dopamine metabolism rather than any single gene that is etiologically important in ADHD. In addition, by comparing the relative effect of different genes in a single group of subjects, one might find that genes affecting a different neurotransmitter, such as norepinephrine, have a greater relative effect on ADHD than dopamine genes.

In the present studies we have used these approaches to answer the following questions: Are dopaminergic, serotonergic or noradrenergic genes more important in ADHD? Are similar or different genes utilized in comorbid disorders such as CD and ODD?

### METHODS

Subjects. The subjects consisted of 326 unrelated, non-Hispanic Caucasians. Of these, 271 had a diagnosis of Tourette syndrome (TS) and 55 were controls. Ten of the subjects were eliminated because of inadequate DNA for the additional analyses. The TS subjects came from the Tourette syndrome Clinic at the City of Hope Medical Center. All meet DSM-IV criteria for TS and all were personally interviewed by David E. Comings. Of the TS subjects 54% met DSM-IV criteria for ADHD. The age of the TS subjects averaged 18.0 years (S.D. 13.2). While the majority were older children and adolescents, 29% were 21 years of age or older. The controls were unrelated adopting or foster parents of the TS probands. These had the advantage that they were of the same geographic, ethnic, and socioeconomic status as the probands and were motivated to participate and could be interviewed at the same time as the probands and were screened to exclude TS, ADHD, ODD, CD and substance abuse. The mean age of the controls was 46.3 years (S.D. 15.38). All TS subjects and controls were given a structured questionnaire based on the Diagnostic Interview Schedule and DSM-IV criteria for a range of disorders. For the ADHD score the questions asked if each of the DSM-IV ADHD criteria during childhood and adolescence were never or rarely present (score=0), occasionally present (score=1) or always present (score=2). The final ADHD score for each subject represented the sum of the above scores for each of the DSM-IV criteria. The subjects were also asked the same questions about each of the DSM-IV ADHD criteria for ODD and CD to provide a quantitative ODD and CD score. Each subject was personally interviewed by David E. Comings to ensure the accuracy of the responses and of the clinical diagnoses.

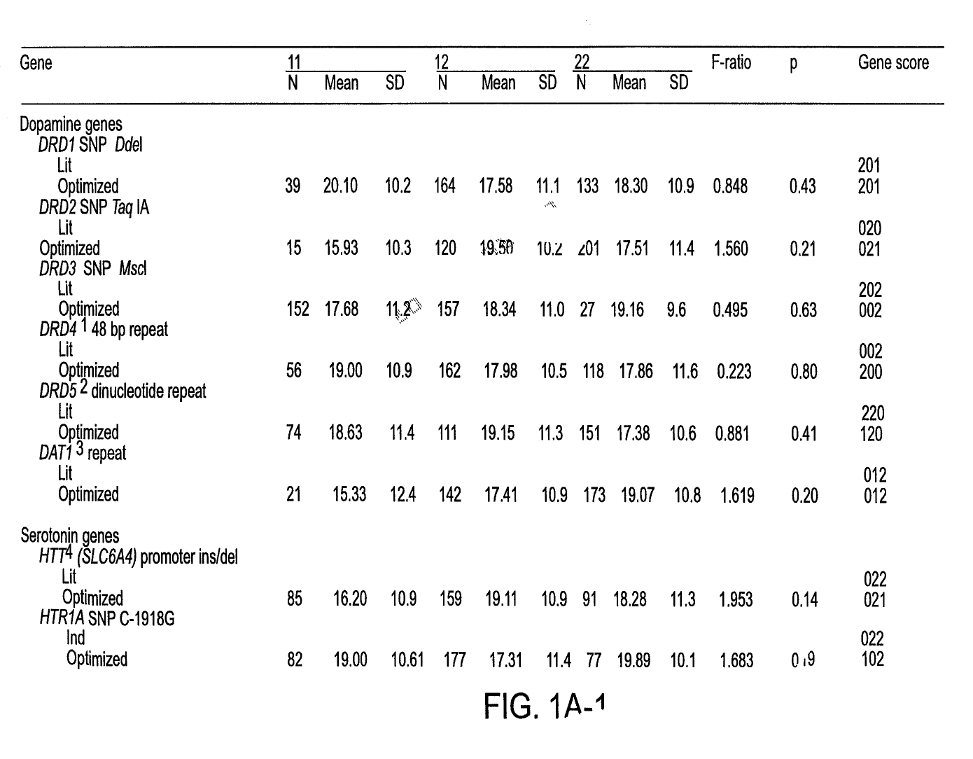

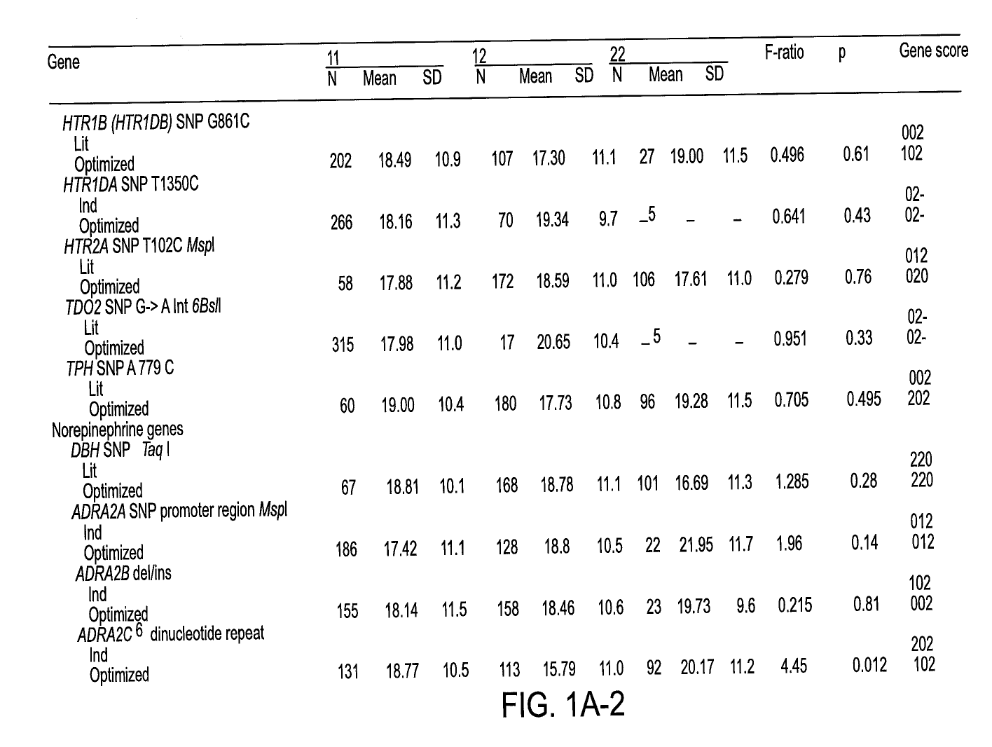

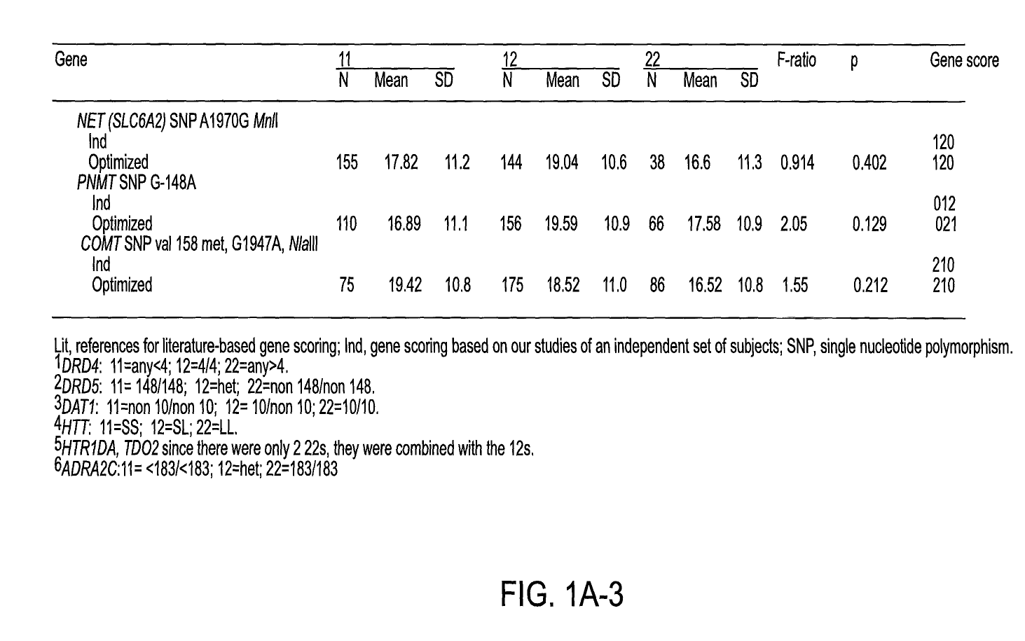

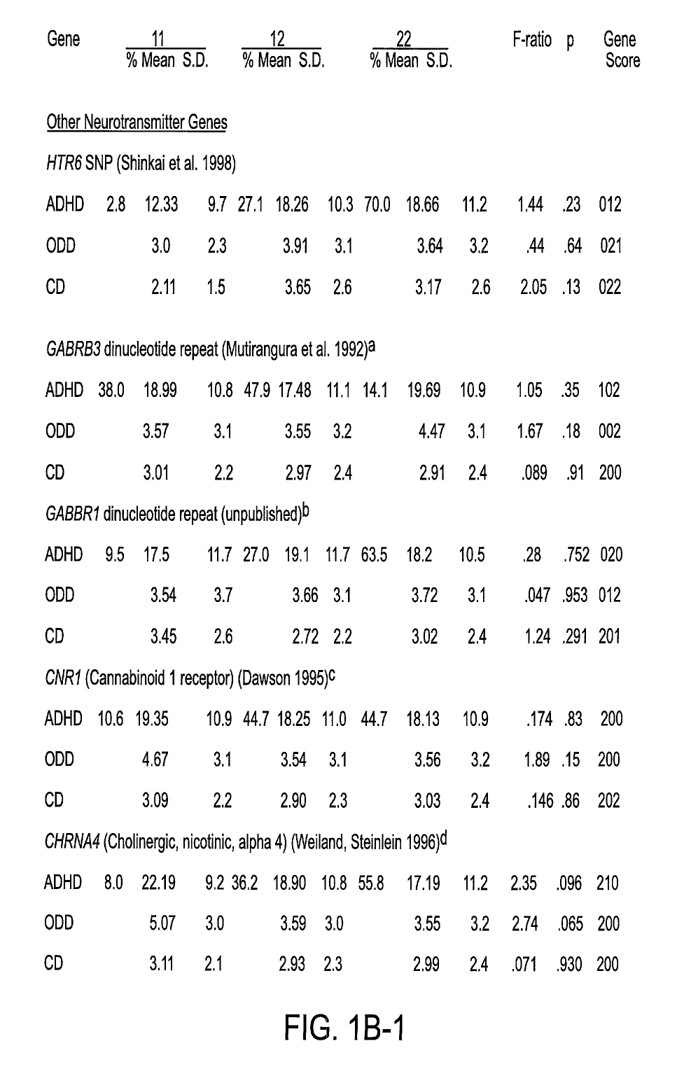

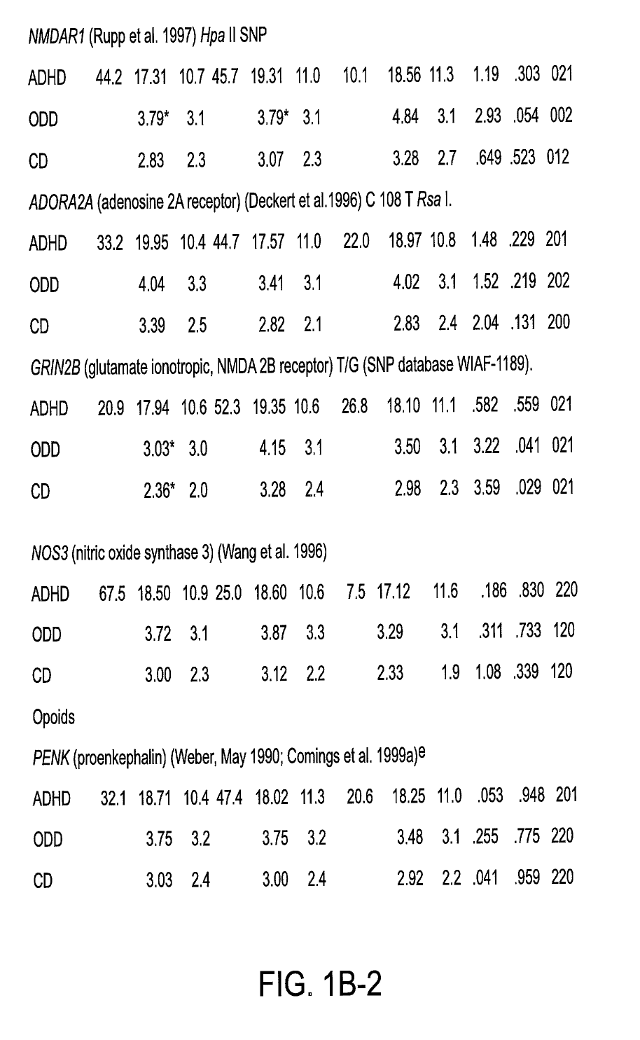

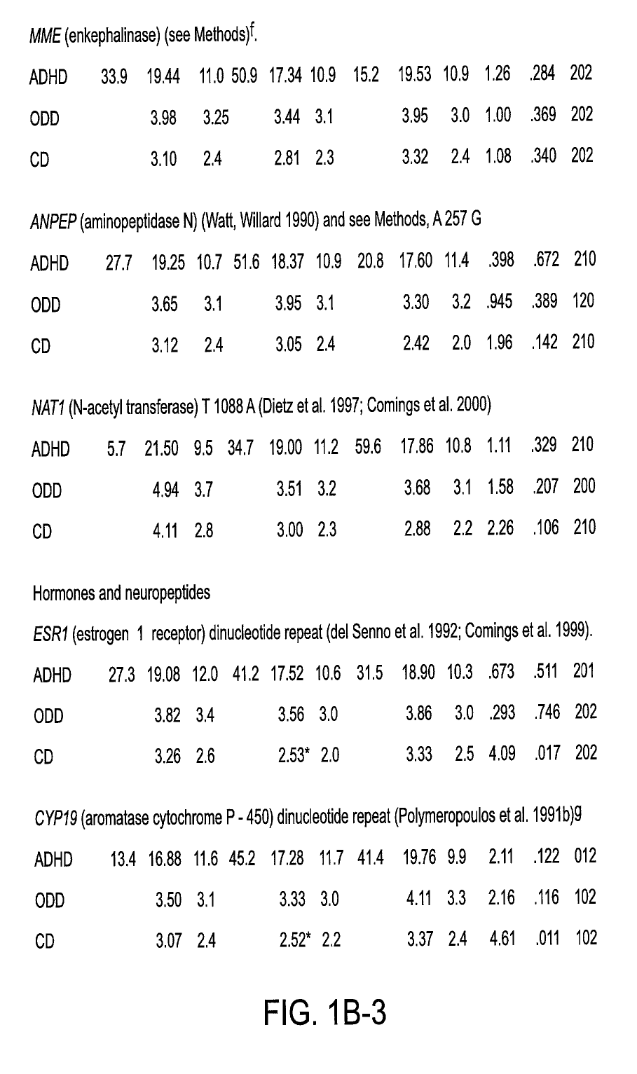

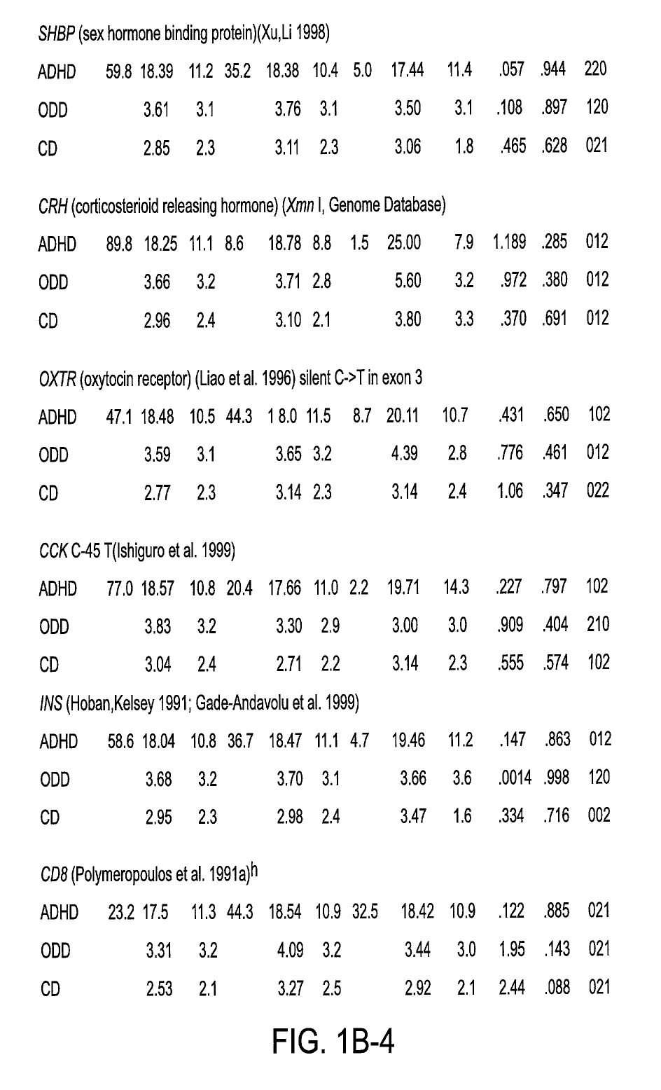

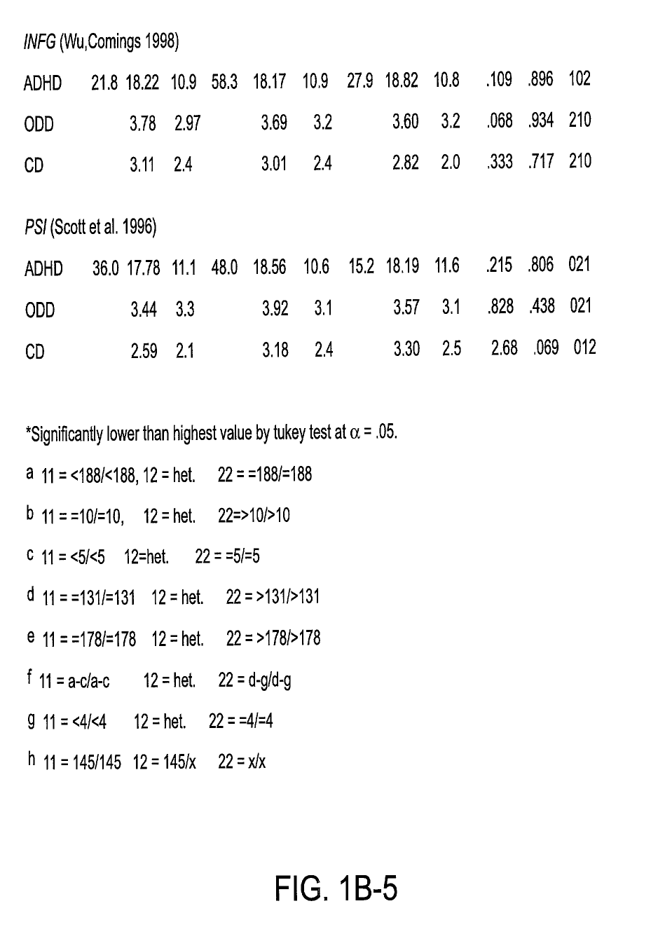

Polymorphisms. The polymorphisms for the first 20 dopamine, serotonin, and norepinephrine genes are provided herein (see FIG. 1a). FIG. 1b describes 22 additional genes, their associated polymorphisms, the results of ANOVA comparing the mean ADHD, ODD and CD scores for the three genotypes, and the resultant gene score based on the ANOVA results. See Scoring of the Genes (supra) for the rationale for the gene scoring and other aspects of the technique. Each gene were scored 0 to 2 based on either prior results in the literature (literature references are set forth in Comings et al., 2000c) about which genotypes were associated with the given phenotype, or based on ANOVA for the three genotypes (11, 12 and 22) on the same subjects used for the multivariate regression analysis. We have termed the latter ‘optimized’ gene scoring and it is used when there is no prior literature for a given gene or a given phenotype. Since all genes are optimized in the same fashion, this allows a comparison of the relative effect of the genes examined. The genes scores in FIG. 1b are optimized genes scores. Since virtually none of these have previously been examined in ADHD, ODD or CD, no literature-based genes scores were available. The following polymorphisms have been developed in our own laboratory and have not been previously published.

MME polymorphism. The sequence of the MME (CD10, neutral endopeptidase 24.11) gene shows a GT repeat in the 5′ region of the gene (Haouas et al. 1995). PCR amplification showed the presence of a polymorphism of this region consisting of 6 MME alleles representing 21 to 26 GT repeats. The use of three forward primers: TTTCAGTATGAATTCCGCAGT (SEQ ID NO:1), GCAGTAAATCATTTTGATATTAAA (SEQ ID NO:2), and TGCTATGAAAAAGATGGAAAATA (SEQ ID NO:3), and a single fluorescent labeled (HEX Amidite, Applied Biosystems, Foster City, Calif.) reverse primer: TGATCCTTTCCTCTTTTGAAT (SEQ ID NO:4), allowed the analysis of the samples on a single well of the Applied Biosystems 373 DNA sequencer.

The PCR reaction was performed under the solution conditions described for the Qiagen PCR Kit (Valencia, Calif.), but not including the Q solution. To a final volume of 14 μl of reaction mixture was added 50 ng of human DNA. The thermocycling protocol consisted of an initial denaturation at 95 degrees C. for 5 minutes; a cycle of 95 degrees for 30 seconds, then one minute at 55 degrees C. and one minute at 72 degrees C. repeated 38 times, ending with an incubation at 72 degrees C. for 5 minutes. Two μl of the 10 fold diluted PCR product was then added to 2.5 ill deionized formamide and 0.5 μl of ROX 500 standard (Applied Biosystems, Foster City, Calif.), denatured for 2 min at 92° C. and loaded on 6% denaturing polyacrylamide gel. The gel was electrophoresed for 5 hours at a constant 25 W. The gel was laser scanned and analyzed using the internal ROX 500 standards present in each lane. The peaks were recognized by Genotyper (version 1.1) based on the color fragments sized by base pair length.

ANPEP polymorphism. Based on the sequencing of the human aminopeptidase (EC 3.4.11.2) gene (Watt, 1990) we identified an A 257 G (gly 86 arg) polymorphism. The PCR primers were forward: CAGGAGAAGAACAAGAACGC (SEQ ID NO:5), reverse: CCTGGCTGAGGGTGTAGTTG (SEQ ID NO:6). The PCR conditions were the same as for the MME polymorphism. These PCR conditions produced a 300 bp product with the polymorphism in the center. When cut with Msp I both products were 150 bp resulting in an enhanced signal. The products were electrophoresed in 2% agarose.

Statistics. Since each gene is treated in the same fashion, their potential role is optimized, i.e., the genes were scored based on ANOVA for the genotypes under examination on the same subjects used for multivariate regression analysis. This allows an examination of the key issue in polygenic disorders, the relative role of each gene or group of genes. For the multivariate regression analysis all the gene scores are added together and the non-significant ones were removed by backward elimination, based on the removal criteria. The pᵢₙ value (p criteria for adding a variable) was set at .1 and the Pₒᵤₜ value (p criteria for removing a variable) was set at 0.2. The SPSS statistical package (SPSS, Inc, Chicago, Ill.) was used.

### Results

FIG. 1(b) lists the results of ANOVA using the continuous traits for ADHD, ODD or CD as the dependent variables and the three genotypes of the 22 additional genes as the independent variables. These results determine how the genes will be scored for the regression analysis. The gene scores for the 22 additional genes plus the 20 earlier described genes were then entered into the regression analysis as independent variables.

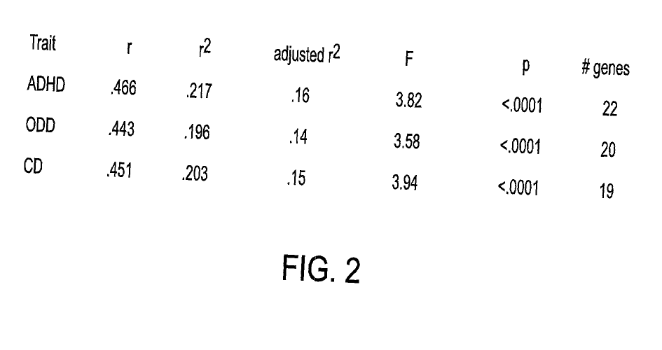

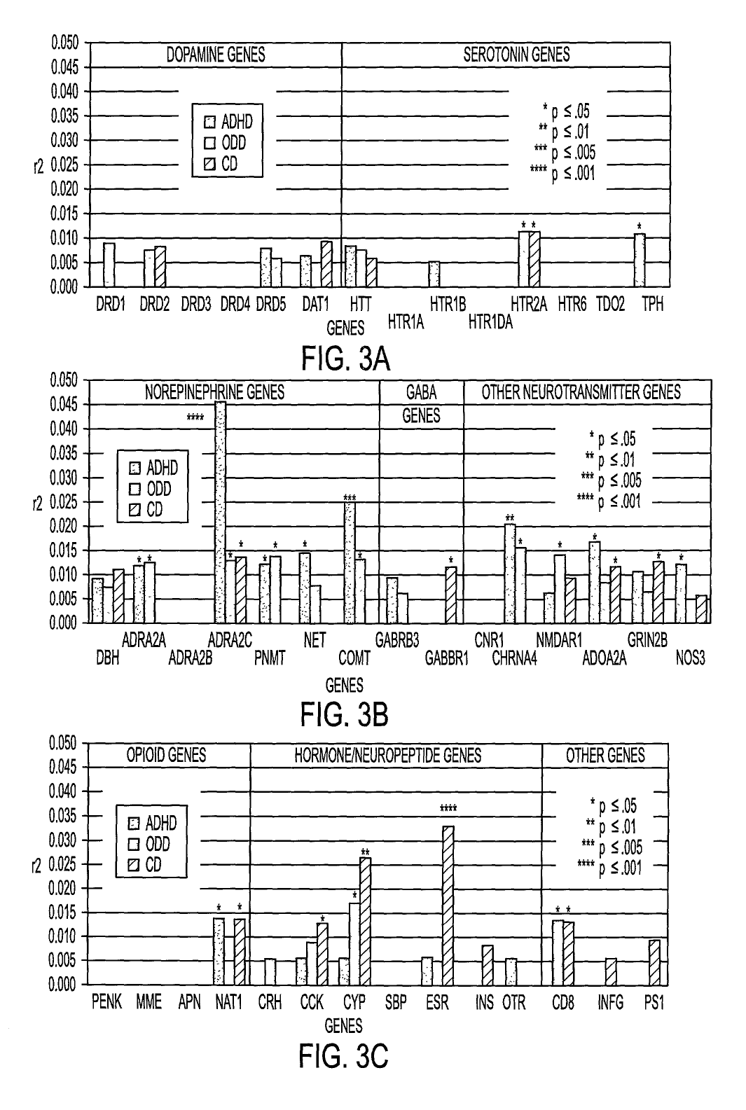

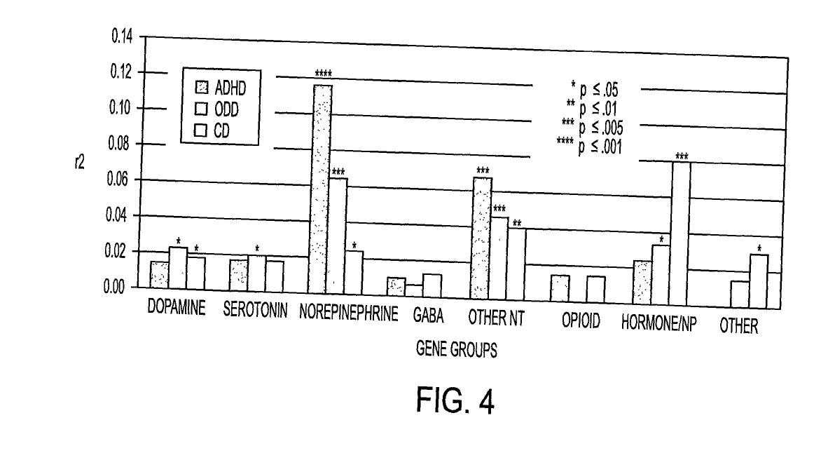

FIG. 2 presents the results for ADHD, ODD, and CD for all 42 genes. The diagram shows the genes and functional groups of genes that were included in the equation, the fraction of the variance (r²) contributed, and the p values that were <0.05. FIG. 2 presents the sum of the r² values for the eight functional groups of genes, based on simply adding the individual r² values from FIGS. 1a and 1b. The p values were based on performing an individual multivariate regression analysis on a given functional group of genes. The p values of those groups that were significant are given. FIG. 2 presents the final r, r², adjusted r^{2,} F, p values, and the number of genes included for ADHD, ODD, and CD, for the 42 genes examined.

### Discussion

ADHD. Despite the addition of 22 more genes, the present study is consistent with the initial study in that the noradrenergic (NE) genes were still the predominant functional group of genes for ADHD. The only group that approached the NE genes in importance were the ‘other neurotransmitter genes.’ Of these the CHRNA4, NMDAR1, and ADOA2A genes were significant at p<0.05, and the NMDAR1 and NOS3 genes were also included in the regression equation. The CHRNA4 gene is a good candidate because of studies showing that nicotine administered by cigarette smoking can enhance attention, arousal, learning and memory (Wesnes 1984; Warburton 1992; Balfour 1996) and reduces the symptoms of ADHD (Coger et al. 1996; Conners et al. 1996; Levin et al. 1996). In addition, recent studies of Wilens et al (Wilens et al. 1999) have shown that ABT-418, a potent agonist at the central cholinergic, nicotinic α₄β₂ receptor, is effective in the treatment of adult ADHD. The ADOA2A gene is candidate for ADHD and CD since caffeine can be useful in the treatment of ADHD and there are many interactions between dopamine (Morelli et al. 1994; Ferré et al. 1997) and adenosine neurons, and knockout mice for the adenosine A2A receptor are aggressive (Ledent et al. 1997). Nitric oxide synthase genes are candidates since nitric oxide may be involved in long term potentiation (Haley et al. 1992; Son et al. 1996) implicated in learning and cognition, and since knockout mice missing the nitric oxide synthase gene were aggressive and hypersexual (Nelson et al. 1995). Studies implicating NMDA and specifically the GRIN2B gene in cognition (Tang et al. 1999), suggest NMDAR1 and GRIN2B as candidate genes for ADHD. Finally, the interactions between anandamide, the endogenous ligand for cannabinoid receptors, and dopamine (Romero et al. 1995), and our previous studies indicating an association between a tetranucleotide repeat polymorphism of the CNR1 gene and drug use (Comings and MacMurray 1997), suggest it as a candidate gene. The observation that 5 of the 6 candidate genes in this group were included in the regression equation, three of which were significant at =0.05, verified that these are useful candidates for studying ADHD. This initial study is strongly dependent upon available polymorphisms. The rapid rate of discovery of new SNPs suggests that within a short period of time many of the genes within the ‘other neurotransporters’ group will emerge as functional groups of their own, e.g., acetyl choline, glutamate, adenosine, nitric oxide functional groups of genes.

The only other new gene that produced a significant individual result was the NAT1 gene. N-acetylation is a common mechanism of detoxifying chemicals and drugs containing arylamines. In humans, two N-acetyl-transferases, NAT-1 and NAT-2 are coded by two closely linked genes, NAT1 and NAT2 on chromosome 8p21.3-23.1 (Hickman D et al. 1994). Polymorphisms of both genes have been linked to rapid and slow acetylation (Grant et al. 1997). We became interested in the NAT1 gene because of its potential role in the metabolism of β-endorphin (Smyth et al. 1979) and our observation of an association between the 1 allele of the NAT1*10 polymorphism and substance abuse (Comings et al. 2000b). Other new genes that were included in the regression equation, but were not individually significant were GABRB3, CCK, CYP19, ESR, and OTR.

ODD. As in the previous study, the dopamine, serotonin and NE genes were all significantly associated with ODD. However, as with ADHD, the ‘other neurotransmitter’ group of genes was the next most important functional group. Within that group the CHRNA4 and NMDAR1 genes were individually significant, and the ADOA2A and GRIN2B genes were included in the regression equation. Most of the reasons for considering these candidate genes for ADHD are also valid for considering them candidate genes for ODD. The other included genes were CRH, CCK, CYP19, and CD8. Since most of these were equally or more involved in CD, they are discussed below.

CD. The group of ‘other neurotransmitter’ genes was also significantly involved in CD, but somewhat less so than for ADHD and ODD. For CD the individually significant genes were the ADOA2A and the GRIN2B. The NMDAR1 and NOS3 genes were also included in the regression equation. The ADOA2A is consistent with the above mentioned aggression in knockout mice missing the ADOA2A gene (Ledent et al. 1997). The inclusion of the GRIN2B gene could be related to the finding that learning disorders are a risk factor for CD in humans (Moffitt 1988).

The most striking aspect of CD was the finding that the ‘hormone/neuropeptide’ group of genes was more significantly involved than any of the neurotransmitter groups of genes. Some but not all studies suggest a correlation between aggressive behavior and plasma testosterone levels (Olweus et al. 1988). We became interested in the role of hormone and neuropeptide genes in CD and other externalizing disorders following our observation that the shorter alleles of the trinucleotide repeats of the androgen receptor (AR) gene were associated with a range of externalizing behaviors (Comings et al. 1999b; Johnson et al. 2000). This was our a priori hypothesis because the shorter alleles have been shown to be correlated with increased expression of the androgen receptor (Choong et al. 1996) and with prostate cancer (Irvine et al. 1995; Giovannucci et al. 1997), an androgen dependent tumor. These observations suggest that the shorter alleles are associated with increased expression of the AR gene. These were the alleles we found to be associated with externalizing behaviors (Comings et al. 1999b; Johnson et al. 2000). In a study involving only normal males, we found the alleles of the GGC trinucleotide repeat of the AR gene were associated with aggressiveness and impulsivity (unpublished). As discussed earlier, we did not examine X-linked genes in the present study since the results are difficult to interpret if the number of females is different in the subjects versus the controls, as was the case here. However, a number of other non-X-linked genes are relevant to the role of androgens in externalizing behaviors. In the brain testosterone is converted to estrogen by aromatase cytochrome P-450 coded by the CYP19 gene and bound by the estrogen 1 receptor coded by the ESRI gene. A third related gene is the sex-hormone binding protein coded by SHBP gene. Other potential hormone and neuropeptide candidate genes were also included. A knockout mouse for the oxytocin gene showed reduced aggression (DeVries et al. 1997) suggesting that one of the genotypes of a polymorphism at this locus could be associated with a relative increase in aggression. The insulin gene was included because of reports of the role of hypoglycemia in aggressive behavior (Roy et al. 1986; Virkkunen et al. 1994). The corticosterone releasing hormone (CRH) is central to stress responses (Nemeroff 1991). Finally, since cholecystokinin (CCK) has been shown to affect locomotion through its effect on striatal dopamine neurons (Fukamauchi et al. 1997; Kobayashi et al. 1996) it was a candidate for ADHD and was one of the included ADHD genes. When the r² values for CD for all of the included hormone and neuropeptide genes were summed they accounted for 8% of the variance of the CD score (p<0.0001).

Because of the studies implicating streptococcal infections in Tourette syndrome and OCD (Trifiletti 1999), we included two genes relevant to immune function, CD1 and INFG. PSI was included since as a risk factor for Alzheimer's disease (Scott et al. 1996) it could be involved in cognitive disorder.

Total variance. FIG. 2 shows the total r, r² and adjusted r² accounted for by the 42 genes examined, and the number of genes that were included in the respective equations. This suggests that for each trait about half of the genes accounted for 19.6 to 21.7 percent of the variance of these traits. However, two factors suggest the total percent of the variance explained is less than this. First, since multivariate regression analysis tends to overestimate the total variance (Cohen 1988), the SPSS program (SPSS, Inc, Chicago, Ill.) provides an adjusted r². The adjusted r² values ranged from 14 to 16 percent of the variance explained. A second factor is that the gene scores were maximized on the same set of subjects used for the regression analyses. While this allows the relative contribution of the different genes or groups of genes to be compared, it enhances random effects and therefore it provides an overestimate of the total variance (Comings et al. 2000a). Combined, these factors suggest that the 19 to 22 genes included in the regression equations account for approximately 10 percent of the variance for the various traits. This is an average of .5% of the variance attributed to each gene. This low effect size in conjunction with the significant genetic heterogeneity of polygenic disorders accounts for the difficulty of identifying the genes involved in complex behavioral disorders using standard linkage approaches and for the frequent difficulty in replication of findings that report results for single genes.

The present study answered the three initial questions First, the genes associated with these other neurotransmitters, especially acetyl choline (CHRNA4), NMDA (NMDAR1), adenosine (ADOA2A), glutamic acid (GRIN2B), and nitric acid (NOS3) were significantly involved with all three traits. Second, CD is significantly associated with the genes for a range of hormones, e.g., AR (Comings et al. 1999b), CCK, CYP19, ESR1, INS. Third, when the number of candidate genes tested was more than doubled, the norepinephrine genes were still the predominant group involved in ADHD.

We propose that the MAA technique, by focusing on the primary characteristic of polygenic disorders, the additive effect of multiple genes, and on the cumulative effect of functionally related groups of genes, provides a powerful approach for the dissection of the genetic basis of complex behavioral disorders.

The present invention provides the information necessary for physicians to select drugs for use in the treatment of ADHD, ODD and CD. With the discovery of the association between the genetic profiles of groups of candidate genes and risk factors for ADHD, ODD and CD, drugs which are known to regulate any of the particular candidate genes associated with ADHD, ODD and/or CD can be selected for the treatment of the above described disorders as well as other disorders.

The present invention also provides a method for screening drug candidates to identify drugs useful for treating ADHD, ODD and CD. Drug screening is performed by comparing the activity of native genes and those described herein in the presence and absence of potential drugs.

The present invention further provides methods for genotyping individuals at risk for ADHD, ODD and/or CD. Such methods analyze the candidate gene in question for known polymorphisms. The genotyping is particularly useful for testing potential drugs for effects on ADHD, ODD and/or CD. The genotyping can also include the identification of other disorders that will respond to drugs that inhibit ADHD activity.

Proof that a given candidate gene is associated with ADHD, ODD and/or CD or risk for ADHD, ODD and/or CD is obtained by finding polymorphisms or sequences in DNA extracted from affected kindred members which create abnormal candidate gene products or abnormal levels of the gene products or which are statistically associated with ADHD, ODD and/or CD. Such susceptibility alleles will co-segregate with the disease in large kindreds. They will also be present at a much higher frequency in non-kindred individuals who have ADHD, ODD and/or CD than in individuals in the general population. The more susceptibility alleles that are present in a person the more likely it is that: i) the person will show symptoms of ADHD, ODD and/or CD; and/or ii) the disease will be more severe.

Detection of point mutations may be accomplished by molecular cloning of the candidate gene's alleles and sequencing the alleles using techniques well known in the art. Also, the gene or portions of the gene may be amplified, e.g., by PCR or other amplification technique, and the amplified gene or amplified portions of the gene may be sequenced.

There are six well known methods for a more complete, yet still indirect, test for confirming the presence of a susceptibility allele: 1) single-stranded conformation analysis (SSCP) (Orita et al., 1989); 2) denaturing gradient gel electrophoresis (DGGE) (Wartell et al., 1990; Sheffield et al., 1989); 3) RNase protection assays (Finkelstein et al., 1990; Kinszler et al., 1991); 4) allele-specific oligonucleotides (ASOs) (Conner et al., 1983); 5) the use of proteins which recognize nucleotide mismatches, such as the E. coli mutS protein (Modrich, 1991); and 6) allele-specific PCR (Ruano and Kidd, 1989). For allele-specific PCR, primers are used which hybridize at their 3′ ends to a particular candidate gene polymorphism or mutation. If the particular polymorphism or mutation is not present, an amplification product is not observed. Amplification Refractory Mutation System (ARMS) can also be used, as disclosed in European Patent Application Publication No. 0332435 and in Newton et al., 1989. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening relatives of an affected individual for the presence of the mutation found in that individual. Other techniques for detecting insertions and deletions as known in the art can be used.

In the first three methods (SSCP, DGGE and RNase protection assay), a new electrophoretic band appears. SSCP detects a band which migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal. In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. In the practice of the present invention, the method involves the use of a labeled riboprobe which is complementary to the human wild-type candidate gene coding sequence. The riboprobe and either mRNA or DNA isolated from the person are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the mRNA or gene but can be a segment of either. If the riboprobe comprises only segment of the mRNA or gene, it will be desirable to use a number of these probes to screen the whole mRNA sequence for mismatches.

In similar fashion, DNA probes can be used to detect mismatches, through enzymatic or chemical cleavage. See, e.g., Cotton et al., 1988; Shenk et al., 1975; Novack et al., 1986. Alternatively, mismatches can be detected by shifts in the electrophoretic mobility of mismatched duplexes relative to matched duplexes. See, e.g., Cariello, 1988. With either riboprobes or DNA probes, the cellular mRNA or DNA which might contain a mutation can be amplified using PCR (see below) before hybridization. Changes in DNA of the candidate gene can also be detected using Southern blot hybridization, especially if the changes are gross rearrangements, such as deletions and insertions.

DNA sequences of the given candidate gene which have been amplified by use of PCR may also be screened using allele-specific probes. These probes are nucleic acid oligomers, each of which contains a region of the gene sequence harboring a known mutation. For example, one oligomer may be about 30 nucleotides in length, corresponding to a portion of the gene sequence. By use of a battery of such allele-specific probes, PCR amplification products can be screened to identify the presence of a previously identified mutation in the gene. Hybridization of allele-specific probes with amplified sequences of the given candidate gene can be performed, for example, on a nylon filter. Hybridization to a particular probe under high stringency hybridization conditions indicates the presence of the same mutation in the tissue as in the allele-specific probe.

The newly developed technique of nucleic acid analysis via microchip technology is also applicable to the present invention. Several papers have been published which use this technique, e.g., Hacia et al., 1996; Shoemaker et al., 1996; Chee et al., 1996; Lockhart et al., 1996; DeRisi et al., 1996; and Lipshutz et al., 1995.

The most definitive test for mutations in a candidate locus is to directly compare genomic candidate gene sequences from patients with those from a control population. Alternatively, one could sequence messenger RNA after amplification, e.g., by PCR, thereby eliminating the necessity of determining the exon structure of the candidate gene.

### Definitions

The present invention employs the following definitions, which are, where appropriate, referenced to genes associated with Reward Deficiency Syndrome (RDS), including ADHD, ODD and CD.

“Amplification of Polynucleotides” utilizes methods such as the polymerase chain reaction (PCR), ligation amplification (or ligase chain reaction, LCR) and amplification methods based on the use of Q-beta replicase. Also useful are strand displacement amplification (SDA), thermophilic SDA, and nucleic acid sequence based amplification (3SR or NASBA). These methods are well known and widely practiced in the art. See, e.g., U.S. Pat. Nos. 4,683,195 and 4,683,202 and Innis et al., 1990 (for PCR); Wu and Wallace, 1989 (for LCR); U.S. Pat. Nos. 5,270,184 and 5,455,166 and Walker et al., 1992 (for SDA); Spargo et al., 1996 (for thermophilic SDA) and U.S. Pat. No. 5,409,818, Fahy et al., 1991 and Compton, 1991 for 3SR and NASBA. Reagents and hardware for conducting PCR are commercially available. Primers useful to amplify sequences from the candidate gene region are preferably complementary to, and hybridize specifically to, sequences in the candidate gene region or in regions that flank a target region therein. Candidate gene sequences generated by amplification may be sequenced directly. Alternatively, but less desirably, the amplified sequence(s) may be cloned prior to sequence analysis. A method for the direct cloning and sequence analysis of enzymatically amplified genomic segments has been described by Scharf et al., 1986.

“Allele” refers to normal alleles of a candidate gene locus as well as alleles of a candidate gene carrying variations that are associated with ADHD, ODD and/or CD. A wild-type allele means a normal allele or an allele comprising only silent mutations. A non-wild-type allele comprises one or more mutations other than a silent mutation. A silent mutation is one which has no effect on the encoded amino acid sequence as compared to the wild-type encoded amino acid sequence.

“Gene Locus”, “Gene”, “Nucleic Acids” or “Polynucleotide” each refer to polynucleotides that are likely to be expressed in normal tissue, certain alleles of which are associated with ADHD, ODD and/or CD or risk of ADHD, ODD and/or CD. The gene locus is intended to include coding sequences, intervening sequences and regulatory elements controlling transcription and/or translation. The gene locus is intended to include all allelic variations of the DNA sequence.

These terms, when applied to a nucleic acid, refer to a nucleic acid which encodes a human polypeptide, fragment, homolog or variant, including, e.g., protein fusions or deletions. The nucleic acids of the present invention will possess a sequence which is either derived from, or substantially similar to a natural peptide-encoding gene or one having substantial homology with a natural peptide-encoding gene or a portion thereof.

The candidate gene or nucleic acid includes normal alleles of the candidate gene, including silent alleles having no effect on the amino acid sequence of the candidate gene's associated polypeptide as well as alleles leading to amino acid sequence variants of the polypeptide that do not substantially affect its function. These terms also include alleles having one or more mutations which adversely affect the function of the polypeptide. A mutation may be a change in the nucleic acid sequence which produces a deleterious change in the amino acid sequence of the polypeptide, resulting in partial or complete loss of function, or may be a change in the nucleic acid sequence which results in the loss of effective expression or the production of aberrant forms of the polypeptide.

A method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Prior to, or as well as being screened for modulation of activity, test substances may be screened for ability to interact with the polypeptide, e.g., in a yeast two-hybrid system (e.g., Bartel et al., 1993; Fields and Song, 1989; Chevray and Nathans, 1992; Lee et al., 1995). This system may be used as a coarse screen prior to testing a substance for actual ability to modulate activity of the polypeptide. Alternatively, the screen could be used to screen test substances for binding to a target protein specific binding partner, or to find mimetics of the target polypeptide.

Following identification of a substance which modulates or affects polypeptide activity, the substance may be further investigated. Furthermore, it may be manufactured and/or used in preparation, i.e., a manufacture or formulation, or a composition such as a medicament, pharmaceutical composition or drug. These may be administered to individuals.

In order to detect the presence of a candidate gene allele predisposing an individual to ADHD, ODD and/or CD, a biological sample such as blood is prepared and analyzed for the presence or absence of susceptibility alleles. In order to detect the presence of a risk of ADHD, ODD and/or CD, a biological sample is prepared and analyzed for the presence or absence of polymorphic or mutant alleles. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis. Suitable diagnostic techniques include those described herein as well as those described in U.S. Pat. Nos. 5,837,492; 5,800,998 and 5,891,628.

Initially, the screening method involves amplification of the relevant target protein sequence. In another preferred embodiment of the invention, the screening method involves a non-PCR based strategy. Such screening methods include two-step label amplification methodologies that are well known in the art. Both PCR and non-PCR based screening strategies can detect target sequences with a high level of sensitivity.

The most popular method used today is target amplification. Here, the target nucleic acid sequence is amplified with polymerases. One particularly preferred method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes.

When the probes are used to detect the presence of the target sequences the biological sample to be analyzed, such as blood or serum, may be treated, if desired, to extract the nucleic acids. The sample nucleic acid may be prepared in various ways to facilitate detection of the target sequence, e.g. denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques known in the art.

Analyte nucleic acid and probe are incubated under conditions which promote stable hybrid formation of the target sequence in the probe with the putative targeted sequence in the analyte. The region of the probes which is used to bind to the analyte can be made completely complementary to the targeted region. Therefore, high stringency conditions are desirable in order to prevent false positives. However, conditions of high stringency are used only if the probes are complementary to regions of the chromosome which are unique in the genome. The stringency of hybridization is determined by a number of factors during hybridization and during the washing procedure, including temperature, ionic strength, base composition, probe length, and concentration of formamide. These factors are outlined in, for example, Maniatis et al., 1982 and Sambrook et al., 1989. Under certain circumstances, the formation of higher order hybrids, such as triplexes, quadraplexes, etc., may be desired to provide the means of detecting target sequences.

Detection of the resulting hybrid, if any, is usually accomplished by the use of labeled probes. Alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation, random priming or kinasing), biotin, fluorescent groups, chemiluminescent groups (e.g., dioxetanes, particularly triggered dioxetanes), enzymes, antibodies, gold nanoparticles and the like. Variations of this basic scheme are known in the art, and include those variations that facilitate separation of the hybrids to be detected from extraneous materials and/or that amplify the signal from the labeled moiety. A number of these variations are reviewed in, e.g., Matthews and Kricka, 1988; Landegren et al., 1988; Mifflin, 1989; U.S. Pat. No. 4,868,105; and in EPO Publication No.225,807.

According to the present invention, a method is also provided of supplying wild-type gene function to a cell which carries a mutant candidate gene allele. Supplying such a function should allow normal functioning of the recipient cells. The wild-type gene or a part of the gene may be introduced into the cell in a vector such that the gene remains extrachromosomal. In such a situation, the gene will be expressed by the cell from the extrachromosomal location. More preferred is the situation where the wild-type gene or a part thereof is introduced into the mutant cell in such a way that it recombines with the endogenous mutant gene present in the cell. Such recombination requires a double recombination event which results in the correction of the gene mutation. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner. Conventional methods are employed, including those described in U.S. Pat. Nos. 5,837,492; 5,800,998 and 5,891,628.

Alternatively, peptides which have target protein activity can be supplied to cells which carry a mutant or missing candidate gene allele. Protein can be produced by expression of the cDNA sequence in bacteria, for example, using known expression vectors. Alternatively, the polypeptide(s) can be extracted from polypeptide-producing mammalian cells. In addition, the techniques of synthetic chemistry can be employed to synthesize the protein. Any of such techniques can provide the preparation of the present invention which comprises the protein. The preparation is substantially free of other human proteins. This is most readily accomplished by synthesis in a microorganism or in vitro. Active molecules can be introduced into cells by microinjection or by use of liposomes, for example. Alternatively, some active molecules may be taken up by cells, actively or by diffusion. Conventional methods are employed, including those described in U.S. Pat. Nos. 5,837,492; 5,800,998 and 5,891,628, each incorporated herein by reference.

The identification of the association between a candidate gene polymorphism/mutations and ADHD, ODD and/or CD permits the early presymptomatic screening of individuals to identify those at risk for developing ADHD, ODD and/or CD or to identify the cause of such disorders. To identify such individuals, the alleles are screened as described herein or using conventional techniques, including but not limited to, one of the following methods: fluorescent in situ hybridization (FISH), direct DNA sequencing, PFGE analysis, Southern blot analysis, single stranded conformation analysis (SSCP), linkage analysis, RNase protection assay, allele-specific oligonucleotide (ASO), dot blot analysis and PCR-SSCP analysis. Also useful is the recently developed technique of DNA microchip technology. Such techniques are described in U.S. Pat. Nos. 5,837,492; 5,800,998 and 5,891,628.

Genetic testing will enable practitioners to identify individuals at risk for ADHD, ODD and/or CD at, or even before, birth. Presymptomatic diagnosis will enable better treatment of these disorders, including the use of existing medical therapies. Genetic testing will also enable practitioners to identify individuals having diagnosed disorders other than those in which the diagnosis results from a candidate gene or group of candidate genes. Genotyping of such individuals will be useful for (a) identifying subtypes of depression that will respond to drugs that inhibit protein activity, (b) identifying subtypes of depression that respond well to placebos versus those that respond better to active drugs and (c) guide new drug discovery and testing.

The invention having been described, it will be apparent to those skilled in the art that the same may be varied in many ways without departing from the spirit and scope of the invention. Any and all such modifications are intended to be included within the scope of the claims.

### LISTS OF REFERENCES

Balfour D J, et al. (1996). Pharmac. Ther. 72:51-81.

Bartel P L, et al. (1993). “Using the 2-hybrid system to detect protein-protein interactions.” In

Cellular Interactions in Development: A Practical Approach, Oxford University Press, pp. 153-179.

Cariello N F (1988). Am. J. Human Genetics 42:726-734.

Chee M, et al. (1996). Science 274:610-614.

Chevray P M and Nathans D N (1992). Proc. Natl. Acad. Sci. USA 89:5789-5793.

Choong C S, et al. (1996). Molec. Endocr. 10:1527-1535.

Coger R W, et al. (1996). J. Psychoactive Drugs 28:229-240.

Cohen J (1988). Statistical Power Analysis for the Behavioral Sciences. Hillsdale, N.J.: Lawrence Erlbaum.

Comings D E, et al. (1991). J. Am. Med. Assn. 266:1793-1800.

Comings D E (1996). Polygenetic inheritance of psychiatric disorders. In: Blum K et al., eds.

Handbook of Psychiatric Genetics. Boca Raton, Fla.: CRC Press.

Comings D E, et al. (1996a). Am. J. Med. Gen. (Neuropsych. Genet.) 67:264-288.

Comings D E and MacMurray J M (1997). Biol. Psychiatry 42:295S-296S.

Comings D E (1998). Mol. Psychiatry 3:21-31.

Comings D E, et al. (1999a). Clin. Genet. 55:160-172.

Comings D E et al. (1999b) Neuroreport, 10:1589-1592.

Comings D E (1999c). Mol. Psychiatry 4:314-316.

Comings D E (1999d). CNS Spectrums 4:15-15.

Comings D E et al. (2000a). Clin. Genetics 57:178-196.

Comings D E et al. (2000b). Neuroreport 11:1227-1230.

Comings D E et al. (2000c). Clin. Genet. 58:31-40.

Compton J (1991). Nature 350:91-92.

Conner B J, et al. (1983). Proc. Natl. Acad. Sci. USA 80:278-282.

Conners C K, et al. (1996). Psychopharmacol. Bull 32:67-73.

Cook E H et al. (1995). Am. J. Hum. Genet. 56:993-998.

Cotten M, et al. (1990). Proc. Natl. Acad. Sci. USA 87:4033-4037.

Daly G, et al (1999). Mol. Psychiatry 4:192-196.

DeRisi J, et al. (1996). Nat. Genet. 14:457-460.

DeVries A C, et al. (1997). J. Neuroendocrinol. 9:363-368.

Fahy E, et al. (1991). PCR Methods Appl. 1:25-33.

Falconer D S (1965). Ann. Hum. Genet. 29:51-71.

Falconer D S (1981). Introduction to Quantitative Genetics, 2d ed., NY: Longman, 1981.

Faraone S V, et al. (1991). Psychol. Med. 21:109-121.

Faraone S V, et al. (1998). Am. J. Med. Gen. (Neuropsych. Genet.) 81:458.

Ferré S, et al. (1997). Trends in Neurosci. 20:482-487.

Fields S and Song O -K (1989). Nature 340:245-246.

Finkelstein J, et al. (1990). Genomics 7:167-172.

Fukamauchi F, et al. (1997). Neuroreport 8:3919-3922.

Gill M, et al. (1997). Mol. Psychiatry 2:311-313.

Gillis J J, et al. (1992). J. Abnorm. Child. Psychol. 20:343-348.

Giovannucci E, et al. (1997). Proc. Natl. Acad. Sci. U.S.A. 94:3320-3323.

Grant D M, et al. (1997). Mutat. Res. 12:61-70.

Hacia J G, et al. (1996). Nature Genetics 14:441-447.

Haley J E, et al. (1992). Neuron 8:211-216.

Haouas H, et al. (1995). Biochem. Biophys. Res. Comm. 207:933-942.

Hickman D, et al.(1994). Biochem. J. 297:441-445.

Hudziak J J, et al. (1997). Am. J. Med. Gen. (Neuropsych. Genet.) 74:567-568.

Innis M A, et al. (1990). PCR Protocols: A Guide to Methods and Applications (Academic Press, San Diego, Calif.).

Irvine R A, et al. (1995). Cancer. Res. 55:1937-1940.

Johnson J P, et al. (2000). Association between the androgen receptor gene (GGC) triplet repeat polymorphism and aggressive, impulsive, and cognitive traits in white male polysubstance abusers. (Submitted).

Kinszler K W, et al. (1991). Science 251:1366-1370.

Kobayashi S, et al. (1996). Neurosci. Lett. 214:61-64.

Lahoste G J, et al. (1996). Mol. Psychiatry 1:121-124.

Landegren U, et al. (1988). Science 242:229-237.

Lau E K, et al. (1997). Am. J. Hum. Genet. 61 :A203.

Ledent C, et al. (1997). Nature 388:674-678.

Levin E D, et al. (1996). Psychopharmacology (Berl). 123:55-63.

Lipshutz R J, et al. (1995). BioTechniques 19:442-447.

Lockhart D J, et al. (1996). Nature Biotechnology 14:1675-1680.

Loehlin J C (1992). Genes and Environment in Personality Development. Newbury Park: Sage, 1992.

Lynch M and Walsh B (1998). Genetics and Analysis of Quantitative Traits. Sutherland, Mass.: Sinaure Associates, Inc., 1998.

Maniatis T, et al. (1982). Molecular Cloning: A Laboratory Manual (Cold Spring Harbor Laboratory, Cold Spring Harbor, N.Y.).

Matthews J A and Kricka L J (1988). Anal. Biochem. 169:1.

Mifflin T E (1989). Clinical Chem. 35:1819-1825.

Modrich P (1991). Ann. Rev. Genet. 25:229-253.

Moffitt T E, et al. (1988). J. Abnormal Psychology 97:330-333.

Morelli M, et al. (1994). Eur. J. Pharmacology 251:21-25.

Nelson R J, et al. (1995). Nature 378:383-386.

Nemeroff CD (1991). Corticotropin-releasing factor. In: C. B. Nemeroff (ed.), Neuropeptides and Psychiatric Disorders (pp. 75-91). Washington, D.C.: American Psychiatric Press.

Newton C R, et al. (1989). Nucl. Acids Res. 17:2503-2516.

Noble E P, et al. (1998). Psychiatry Res. 81:133-147.

Novack D F, et al. (1986). Proc. Natl. Acad. Sci. USA 83:586-590.

Olweus D, et al. (1988). Psychosomatic Med. 50:261-272.

Orita M, et al. (1989). Proc. Natl. Acad. Sci. USA 86:2766-2770.

Pastinen T, et al. (1998). Hum. Mol. Genet. 7:1453-1462.

Romero J, et al. (1995). Life Sci. 56:2033-2040.

Rowe D C, et al. (1998). Mol. Psychiatry 3:419-426.

Roy A, et al. (1986). Psychopharmacol. Bull. 22:661-665.

Ruano G and Kidd K K (1989). Nucl. Acids Res. 17:8392.

Sambrook J, et al. (1989). Molecular Cloning: A Laboratory Manual, 2nd Ed. (Cold Spring Harbor Laboratory, Cold Spring Harbor, N.Y.).

Scott W K, et al. (1996). Lancet 347:1185-1186.

Sheffield V C, et al. (1989). Proc. Natl. Acad. Sci. USA 86:232-236.

Shenk T E, et al. (1975). Proc. Natl. Acad. Sci. USA 72:989-993.

Sherman D K, et al. (1997a). J. Am. Acad. Child. Adolesc. Psychiatry. 36:745-753.

Sherman D K, et al. (1997b). Am. J. Psychiatry 154:532-535.

Shoemaker D D, et al. (1996). Nature Genetics 14:450-456.

Smyth D G, et al. (1979). Nature 279:252-254.

Son H, et al. (1996). Cell 87:1015-1023.

Spargo C A, et al. (1996). Mol. Cell. Probes 10:247-256.

Stevenson J (1992). Behav. Genet. 22:337-344.

Stevenson J, et al. (1993). J. Child. Psychol. Psychiatry 34:1137-1152.

Tang Y -P, et al. (1999). Nature 401:63-69.

Trifiletti R R, et al. (1999). Child Adolesc. Psychiatr. Clin. N. Am. 8:767-775.

Virkkunen M, et al. (1994). Arch. Gen. Psychiatry 51:20-27.

Walker G T, et al. (1992). Nucl. Acids Res. 20:1691-1696.

Warburton D M (1992). Prog. Neuropsychopharmacol. Biol. Psychiatry 16:181 -191.

Wartell R M, et al. (1990). Nucl. Acids Res. 18:2699-2705.

Watt V M, et al. (1990). Hum. Genet. 85:651-654.

Wesnes K and Warburton DM (1984). Smoking, nicotine and human performance. In: Balfour, D J K, Nicotine and the Tobacco Smoking Habit, Section 114. The International Encyclopedia of Pharmacology and Therapeutics (pp. 133-152). New York: Pergamon Press.

Wilens T E, et al. (1999). American Journal of psychiatry, 156(12):193 1-1937.

Wu D Y and Wallace R B (1989). Genomics 4:560-569.

### Patents and Patent Applications

EPO Publication No. 225,807.

U.S. Pat. No. 4,683,195.

U.S. Pat. No. 4,683,202.

U.S. Pat. No. 4,868,105.

U.S. Pat. No. 5,270,184.

U.S. Pat. No. 5,409,818.

U.S. Pat. No. 5,455,166.

U.S. Pat. No. 5,800,998.

U.S. Pat. No. 5,837,492.

U.S. Pat. No. 5,891,628.

What is claimed is:

1. A method of determining whether a subject is at risk for attention deficit hyperactivity disorder (ADHD), wherein said method comprises determining whether said subject's genome comprises a non-wild-type allele of at least one gene selected from a first group consisting of TPH, PNMT, ADOA2A, NOS3 and NAT1, wherein the presence of said non-wild-type allele of said gene indicates an increased risk of said subject having ADHD as compared to a person with a wild-type allele of each of said genes.

2. The method of claim 1, wherein said method comprises determining for each of said genes whether a non-wild-type allele is present.

3. The method of claim 1, wherein the presence of more than one of said genes comprising a non-wild-type allele indicates a greater risk for ADHD.

4. The method of claim 1, wherein said method further comprises determining whether said subject's genome comprises a non-wild-type allele of at least one gene selected from a second group consisting of ADRA2A, ADRA2C, NET, COMT and CHRNA4, wherein the presence of said non-wild-type allele of said gene from said second group indicates an increased risk of said subject having ADHD as compared to a person with a wild-type allele of each of said genes from said second group.

5. The method of claim 4, wherein said method comprises determining for each of said genes of said second group whether a non-wild-type allele is present.

6. The method of claim 4, wherein said method comprises determining for each of said genes of said first group whether a non-wild-type allele is present.

7. The method of claim 4, wherein said method comprises determining for each of said genes of said first group and for each of said genes of said second group whether a non-wild-type allele is present.

8. The method of claim 4, wherein the presence of more than one non-wild-type allele indicates a greater risk for ADHD.

9. A method for developing a polygenic assay that is diagnostic for attention deficit hyperactivity disorder, comprising the steps of:
(a) identifying a trait that is to be studied;
(b) creating a scale measuring the severity of the trait to be studied;
(c) selecting at least one candidate gene that may contribute to said trait, wherein said gene is selected from the group consisting of TPH, PNMT, ADOA2A, NOS3 and NAT1 ;
(d) identifying at least one polymorphism associated with said candidate gene; and
(e) correlating allelic patterns of said polymorphism with said scale;
wherein allelic patterns that are positively associated with said trait are added, to form a polygenic assay that is diagnostic.

10. A method of determining a treatment modality for a human subject suspected of having attention deficit hyperactivity disorder (ADHD), wherein said method comprises determining whether said subject's genome comprises a non-wild-type allele of at least one gene selected from a first group consisting of TPH, PNMT, ADOA2A, NOS3 and NAT1, wherein said treatment modality is based upon which of said genes are non-wild-type.

11. The method of claim 10, wherein said method comprises determining for each of said genes whether a non-wild-type allele is present.

12. The method of claim 10, wherein said method further comprises determining whether said subject's genome comprises a non-wild-type allele of at least one gene selected from a second group consisting of ADRA2A, ADRA2C, NET, COMT and CHRNA4.

13. The method of claim 12, wherein said method comprises determining for each of said genes of said second group whether a non-wild-type allele is present.

14. The method of claim 12, wherein said method comprises determining for each of said genes of said first group whether a non-wild-type allele is present.

15. The method of claim 12, wherein said method comprises determining for each of said genes of said first group and for each of said genes of said second group whether a non-wild-type allele is present.

16. A method of determining whether a subject is at risk for oppositional defiant disorder (ODD), wherein said method comprises determining whether said subject's genome comprises a non-wild-type allele of at least one gene selected from a first group consisting of HTR2A, PNMT and CD8, wherein the presence of said non-wild-type allele of said gene indicates an increased risk of said subject having ODD as compared to a person with a wild-type allele of each of said genes.

17. The method of claim 16, wherein said method comprises determining for each of said genes whether a non-wild-type allele is present.

18. The method of claim 16, wherein the presence of more than one of said genes comprising a non-wild-type allele indicates a greater risk for ODD.

19. The method of claim 16, wherein said method further comprises determining whether said subject's genome comprises a non-wild-type allele of at least one gene selected from a second group consisting of ADRA2A, ADRA2C, COMT, CHRNA4, NMDAR1 and CYP, wherein the presence of said non-wild-type allele of said gene from said second group indicates an increased risk of said subject having ODD as compared to a person with a wild-type allele of each of said genes.

20. The method of claim 19, wherein said method comprises determining for each of said genes of said second group whether a non-wild-type allele is present.

21. The method of claim 19, wherein said method comprises determining for each of said genes of said first group whether a non-wild-type allele is present.

22. The method of claim 19, wherein said method comprises determining for each of said genes of said first group and for each of said genes of said second group whether a non-wild-type allele is present.

23. The method of claim 19, wherein the presence of more than one non-wild-type allele indicates a greater risk for ODD.

24. A method for developing a polygenic assay that is diagnostic for oppositional defiant disorder, comprising the steps of:
(a)identifying the trait that is to be studied;
(b) creating a scale measuring the severity of the trait to be studied;
(c) selecting at least one candidate gene that may contribute to said trait, wherein said gene is selected from the group consisting of HTR2A, PNMT and CD8;
(d) identifying at least one polymorphism associated with said candidate gene; and
(e) correlating allelic patterns of said polymorphism with said scale;
wherein allelic patterns that are positively associated with said trait are added, to form a polygenic assay that is diagnostic.

25. A method of determining a treatment modality for a human subject suspected of having oppositional defiant disorder (ODD), wherein said method comprises determining whether said subject's genome comprises a non-wild-type allele of at least one gene selected from a first group consisting of HTR2A, PNMT and CD8, wherein said treatment modality is based upon which of said genes are non-wild-type.

26. The method of claim 25, wherein said method comprises determining for each of said genes whether a non-wild-type allele is present.

27. The method of claim 25, wherein said method further comprises determining whether said subject's genome comprises a non-wild-type allele of at least one gene selected from a second group consisting of ADRA2A, ADRA2C, COMT, CHRNA4, NMDAR1 and CYP.

28. The method of claim 27, wherein said method comprises determining for each of said genes of said second group whether a non-wild-type allele is present.

29. The method of claim 27, wherein said method comprises determining for each of said genes of said first group whether a non-wild-type allele is present.

30. The method of claim 27, wherein said method comprises determining for each of said genes of said first group and for each of said genes of said second group whether a non-wild-type allele is present.

31. A method of determining whether a subject is at risk for conduct disorder (CD), wherein said method comprises determining whether said subject's genome comprises a non-wild-type allele of at least one gene selected from a first group consisting of HTR2A, GABBR1, ADOA2A, GRIN2B, NAT1, CCK, CYP, ESR and CD8, wherein the presence of said non-wild-type allele of said gene indicates an increased risk of said subject having CD as compared to a person with a wild-type allele of each of said genes.

32. The method of claim 31, wherein said method comprises determining for each of said genes whether a non-wild-type allele is present.

33. The method of claim 31, wherein the presence of more than one of said genes comprising a non-wild-type allele indicates a greater risk for CD.

34. The method of claim 31, wherein said method further comprises determining whether said subject's genome comprises a non-wild-type allele of at least one gene selected from a second group consisting of ADRA2C. and PSI, wherein the presence of said non-wild-type allele of said gene from said second group indicates an increased risk of said subject having CD as compared to a person with a wild-type allele of each of said genes.

35. The method of claim 34, wherein said method comprises determining for each of said genes of said second group whether a non-wild-type allele is present.

36. The method of claim 34, wherein said method comprises determining for each of said genes of said first group whether a non-wild-type allele is present.

37. The method of claim 34, wherein said method comprises determining for each of said genes of said first group and for each of said genes of said second group whether a non-wild-type allele is present.

38. The method of claim 34, wherein the presence of more than one non-wild-type allele indicates a greater risk for CD.

39. A method for developing a polygenic assay that is diagnostic for conduct disorder, comprising the steps of.
(a) identifying the trait that is to be studied;
(b) creating a scale measuring the severity of the trait to be studied;
(c) selecting at least one candidate gene that may contribute to said trait, wherein said gene is selected from the group consisting of HTR2A, GABBR1, ADOA2A, GRJN2B, NAT1, CCK, CYP, ESR and CD8;
(d) identifying at least one polymorphism associated with said candidate gene; and
(e) correlating allelic patterns of said polymorphism with said scale;
wherein allelic patterns that are positively associated with said trait are added, to form a polygenic assay that is diagnostic.

40. A method of determining a treatment modality for a human subject suspected of having conduct disorder (CD), wherein said method comprises determining whether said subject's genome comprises a non-wild-type allele of at least one gene selected from a first group consisting of HTR2A, GABBR1, ADOA2A, GRIN2B, NAT1, CCK, CYP, ESR and CDS, wherein said treatment modality is based upon which of said genes are non-wild-type.

41. The method of claim 40, wherein said method comprises determining for each of said genes whether a non-wild-type allele is present.

42. The method of claim 40, wherein said method further comprises determining whether said subject's genome comprises a non-wild-type allele of at least one gene selected from a second group consisting of ADRA2C. and PSI.

43. The method of claim 42, wherein said method comprises determining for each of said genes of said second group whether a non-wild-type allele is present.

44. The method of claim 42, wherein said method comprises determining for each of said genes of said first group whether a non-wild-type allele is present.

45. The method of claim 42, wherein said method comprises determining for each of said genes of said first group and for each of said genes of said second group whether a non-wild-type allele is present.

46. A method of screening drug candidates for a potentially useful drug candidate for treating ADHD, said method comprising the steps of:
a) measuring an activity of a wild-type protein selected from the group consisting of TPH, PNMT, ADOA2A, NOS3 and NAT1;
b) measuring an activity of a non-wild-type protein selected from the group consisting of TPH, PNMT, ADOA2A, NOS3 and NAT1, wherein the activity measured in step (b) is the same activity as measured in step (a) and wherein the protein of step (b) is a non-wild-type allele of the protein of step (a);
c) measuring in the presence of a drug candidate an activity of a non-wild-type protein selected from the group consisting of TPH, PNMT, ADOA2A, NOS3 and NAT1, wherein the activity measured in step (c) is the same as the activity measured in step (a) and wherein the protein of step (c) is a non-wild-type allele of the protein of step (a); and
d) comparing the measured activities of: i) step (a) with step (b) and ii) step (a) with step (c);
wherein if the measured activity of step (c) is closer to the measured activity of step (a) than is the measured activity of step (b) as compared with step (a) then said drug candidate of step (c) is a potentially useful drug candidate for treating ADHD.

47. A method of screening drug candidates for a potentially useful drug candidate for treating ODD, said method comprising the steps of:
a) measuring an activity of a wild-type protein selected from the group consisting of HTR2A, PNMT and CD8;
b) measuring an activity of a non-wild-type protein selected from the group consisting of HTR2A, PNMT and CD8, wherein the activity measured in step (b) is the same activity as measured in step (a) and wherein the protein of step (b) is a non-wild-type allele of the protein of step (a);
c) measuring in the presence of a drug candidate an activity of a non-wild-type protein selected from the group consisting of HTR2A, PNMT and CD8, wherein the activity measured in step (c) is the same as the activity measured in step (a) and wherein the protein of step (c) is a non-wild-type allele of the protein of step (a); and
d) comparing the measured activities of: i) step (a) with step (b) and ii) step (a) with step (c);
wherein if the measured activity of step (c) is closer to the measured activity of step (a) than is the measured activity of step (b) as compared with step (a) then said drug candidate of step (c) is a potentially useful drug candidate for treating ODD.

48. A method of screening drug candidates for a potentially useful drug candidate for treating CD, said method comprising the steps of:
a) measuring an activity of a wild-type protein selected from the group consisting of HTR2A, GABBR1, ADOA2A, GRIN2B, NATI, CCK, CYP, ESR and CD8;
b) measuring an activity of a non-wild-type protein selected from the group consisting of HTR2A, GABBR1, ADOA2A, GRIN2B, NAT1, CCK, CYP, ESR and CD8, wherein the activity measured in step (b) is the same activity as measured in step (a) and wherein the protein of step (b) is a non-wild-type allele of the protein of step (a);
c) measuring in the presence of a drug candidate an activity of a non-wild-type protein selected from the group consisting of HTR2A, GABBR1, ADOA2A, GRIN2B, NAT1, CCK, CYP, ESR and CD8, wherein the activity measured in step (c) is the same as the activity measured in step (a) and wherein the protein of step (c) is a non-wild-type allele of the protein of step (a); and
d) comparing the measured activities of: i) step (a) with step (b) and ii) step (a) with step (c);
wherein if the measured activity of step (c) is closer to the measured activity of step (a) than is the measured activity of step (b) as compared with step (a) then said drug candidate of step (c) is a potentially useful drug candidate for treating CD.

49. A method of treating a subject for the symptoms of ADHD, said method comprising administering to said subject one or more wild-type genes selected from the group consisting of TPH, PNMT, ADOA2A, NOS3 and NAT1, wherein said one or more wild-type genes are expressed in said subject and further wherein said subject's genome comprises a non-wild-type allele of said one or more wild-type genes which are administered.

50. A method of treating a subject for the symptoms of ODD, said method comprising administering to said subject one or more wild-type genes selected from the group consisting of HTR2A, PNMT and CD8, wherein said one or more wild-type genes are expressed in said subject and further wherein said subject's genome comprises a non-wild-type allele of said one or more wild-type genes which are administered.

51. A method of treating a subject for the symptoms of CD, said method comprising administering to said subject one or more wild-type genes selected from the group consisting of HTR2A, GABBR1, ADOA2A, GRJN2B, NAT1, CCK, CYP, ESR and CD8, wherein said one or more wild-type genes are expressed in said subject and further wherein said subject's genome comprises a non-wild-type allele of said one or more wild-type genes which are administered.

52. A method of treating a subject for the symptoms of ADHD, said method comprising administering to said subject one or more wild-type proteins selected from the group consisting of TPH, PNMT, ADOA2A, NOS3 and NAT1, wherein said subject's genome comprises a non-wild-type gene encoding an allelic version of said one or more wild-type proteins which are administered.

53. A method of treating a subject for the symptoms of ODD, said method comprising administering to said subject one or more wild-type proteins selected from the group consisting of HTR2A, PNMT and CD8, wherein said subject's genome comprises a non-wild-type gene encoding an allelic version of said one or more wild-type proteins which are administered.

54. A method of treating a subject for the symptoms of CD, said method comprising administering to said subject one or more wild-type proteins selected from the group consisting of HTR2A, GABBR1, ADOA2A, GRIN2B, NAT1, CCK, CYP, ESR and CD8, wherein said subject's genome comprises a non-wild-type gene encoding an allelic version of said one or more wild-type proteins which are administered.


SEQUENCE LISTING (6 sequences)

SEQ ID NO: 1 - DNA, 21 bp, Homo sapiens
tttcagtatgaattccgcagt

SEQ ID NO: 2 - DNA, 24 bp, Homo sapiens
gcagtaaatcattttgatattaaa

SEQ ID NO: 3 - DNA, 23 bp, Homo sapiens
tgctatgaaaaagatggaaaata

SEQ ID NO: 4 - DNA, 21 bp, Homo sapiens
tgatcctttcctcttttgaat

SEQ ID NO: 5 - DNA, 20 bp, Homo sapiens
caggagaagaacaagaacgc

SEQ ID NO: 6 - DNA, 20 bp, Homo sapiens
cctggctgagggtgtagttg